(19) United States
(12) Patent Application Publication
(10) Pub. No.: US 2014/0058375 A1
(43) Pub. Date: Feb. 27, 2014

(54) HIGH RESOLUTION MAP AND ABLATE CATHETER

(71) Applicant: Boston Scientific Scimed, Inc., Maple Grove, MN (US)

(72) Inventor: Josef V. Koblish, Sunnyvale, CA (US)

(21) Appl. No.: 13/973,631

(22) Filed: Aug. 22, 2013

Related U.S. Application Data
(60) Provisional application No. 61/691,853, filed on Aug. 22, 2012.

(51) Int. Cl. A61B 18/18

(52) U.S. Cl. CPC A61B 18/18
USPC 606/33

(57) ABSTRACT

A medical system includes a medical probe including an elongated member having a distal end, a metallic electrode mounted to the distal end of the elongated member, and a plurality of microelectrodes embedded within, and electrically insulated from, the metallic electrode. A radio frequency (RF) ablation source is configured to deliver RF ablation energy to the metallic electrode. A filter circuit is electrically connected to the microelectrodes such that the filter circuit receives electrical signals from the microelectrodes. The filter circuit is configured to filter signal components induced by the RF ablation energy from the electrical signals. A mapping processor electrically is coupled to the filter circuit and configured to receive and process the filtered electrical signals.

### CROSS-REFERENCE TO RELATED APPLICATION

This application claims the benefit of Provisional Application No. 61/691,853, filed Aug. 22, 2012, which is incorporated herein by reference in its entirety.

### FIELD

The present disclosure generally relates to systems and methods for providing therapy to a patient. More particularly, the present disclosure relates to systems and methods for mapping and ablating tissue within the heart of the patient.

### BACKGROUND

Physicians make use of catheters in medical procedures to gain access into interior regions of the body to ablate targeted tissue regions. It is important for the physician to be able to precisely locate the catheter and control its emission of energy within the body during these tissue ablation procedures. For example, in electrophysiological therapy, ablation is used to treat cardiac rhythm disturbances in order to restore the normal function of the heart.

Normal sinus rhythm of the heart begins with the sinoatrial node (or “SA node”) generating a depolarization wave front that propagates uniformly across the myocardial tissue of the right and left atria to the atrioventricular node (or “AV node”). This propagation causes the atria to contract in an organized manner to transport blood from the atria to the ventricles. The AV node regulates the propagation delay to the atrioventricular bundle (or “His bundle”), after which the depolarization wave front propagates uniformly across the myocardial tissue of the right and left ventricles, causing the ventricles to contract in an organized manner to transport blood out of the heart. This conduction system results in the described, organized sequence of myocardial contraction leading to a normal heartbeat.

Sometimes, aberrant conductive pathways develop in heart tissue, which disrupt the normal path of depolarization events. For example, anatomical obstacles in the atria or ventricles can disrupt the normal propagation of electrical impulses. These anatomical obstacles (called “conduction blocks”) can cause the depolarization wave front to degenerate into several circular wavelets that circulate about the obstacles. These wavelets, called “reentry circuits,” disrupt the normal activation of the atria or ventricles. As a further example, localized regions of ischemic myocardial tissue may propagate depolarization events slower than normal myocardial tissue. The ischemic region, also called a “slow conduction zone,” creates errant, circular propagation patterns, called “circus motion.” The circus motion also disrupts the normal depolarization patterns, thereby disrupting the normal contraction of heart tissue. The aberrant conductive pathways create abnormal, irregular, and sometimes life-threatening heart rhythms, called arrhythmias. An arrhythmia can take place in the atria, for example, as in atrial tachycardia (AT), atrial fibrillation (AFIB), or atrial flutter (AF). The arrhythmia can also take place in the ventricle, for example, as in ventricular tachycardia (VT).

In treating these arrhythmias, it is essential that the location of the sources of the aberrant pathways (called substrates) be located. Once located, the tissue in the substrates can be destroyed, or ablated, by heat, chemicals, or other means of creating a lesion in the tissue, or otherwise can be electrically isolated from the normal heart circuit. Electrophysiology therapy involves locating the aberrant pathways via a mapping procedure, and forming lesions by soft tissue coagulation on the endocardium (the lesions being 1 to 15 cm in length and of varying shape) using an ablation catheter to effectively eliminate the aberrant pathways. In certain advanced electrophysiology procedures, as part of the treatment for certain categories of atrial fibrillation, it may be desirable to create a curvilinear lesion around or within the ostia of the pulmonary veins (PVs), and a linear lesion connecting one or more of the PVs to the mitral valve annulus. Such curvilinear lesion may be formed as far out from the PVs as possible to ensure that the conduction blocks associated with the PVs are indeed electrically isolated from the active heart tissue.

Primarily due to the relatively large size of tip electrodes, some catheter designs may detect far field electrical activity (i.e., the ambient electrical activity away from the recording electrode(s)), which can negatively affect the detection of local electrical activity. That is, due to the relatively large size of the tip electrode and the distance from the next ring electrode, the resulting electrical recordings are signal averaged and blurred, and thus not well-defined. This far-field phenomenon becomes more exaggerated, thereby decreasing the mapping resolution, as the length of distal tip electrode increases.

Thus, the electrical activity measured by such catheters does not always provide a physician with enough resolution to accurately identify an ablation site and or provide the physician with an accurate portrayal of the real position of the tip electrode, thereby causing the physician to perform multiple ablations in several areas, or worse yet, to perform ablations in locations other than those that the physician intends.

In addition, many significant aspects of highly localized electrical activity may be lost in the far-field measurement. For example, the high frequency potentials that are encountered around pulmonary veins or fractioned EGMs associated with atrial fibrillation triggers may be lost. Also, it may be difficult to determine the nature of the tissue with which the tip electrode is in contact, or whether the tip electrode is in contact with tissue at all, since the far-field measurements recorded by the tip electrode may indicate electrical activity within the myocardial tissue even though the tip electrode is not actually in contact with the endocardial tissue.

For example, it may be very important to ascertain whether the tip electrode is in contact with endocardial tissue or venous tissue during an ablation procedure. This becomes especially significant when ablating in and around the ostia of the pulmonary veins, since ablation within the pulmonary veins, themselves, instead of the myocardial tissue, may cause stenosis of the pulmonary veins. However, the far field measurements taken by the tip electrode may indicate that the tip electrode is in contact with endocardial tissue, when in fact, the tip electrode is in contact with venous tissue. As another example, it may be desirable to ascertain lesion formation by measuring the electrical activity of the tissue in contact with the tip electrode (i.e., the lack of electrical activity indicates ablated tissue, whereas the presence of electrical activity indicates live tissue). However, due to the far-field measurements, electrical activity may be measured from nearby live tissue, even though the tip electrode is actually in contact with ablated tissue.

### SUMMARY

Disclosed herein is a filtering circuit configured to reduce or eliminate signal components induced by RF ablation energy on an ablation electrode from electrical signals received at mapping microelectrodes, as well as medical systems including the filtering circuit.

In Example 1, a medical system includes a medical probe including an elongated member having a distal end, a metallic electrode mounted to the distal end of the elongated member, and a plurality of microelectrodes embedded within, and electrically insulated from, the metallic electrode. A radio frequency (RF) ablation source is configured to deliver RF ablation energy to the metallic electrode. A filter circuit is electrically connected to the microelectrodes such that the filter circuit receives electrical signals from the microelectrodes. The filter circuit is configured to filter signal components induced by the RF ablation energy from the electrical signals. A mapping processor electrically is coupled to the filter circuit and configured to receive and process the filtered electrical signals.

In Example 2, the medical system according to Example 1, wherein the filter circuit comprises a plurality of filters each associated with one of the plurality of microelectrodes.

In Example 3, the medical system according to either Example 1 or Example 2, wherein the filter circuit is configured to filter frequency components from the electrical signals in the range of about 30-600 kHz.

In Example 4, the medical system according to any of Examples 1-3, wherein the filter circuit comprises one or more passive filters.

In Example 5, the medical system according to any of Examples 1-4, wherein the filter circuit comprises one or more digital filters.

In Example 6, the medical system according to any of Examples 1-5, wherein the medical probe further comprises one or more mapping ring electrodes, and wherein the one or more mapping ring electrodes are electrically connected to the filter circuit to filter RF ablation energy induced components from signals from the mapping ring electrodes.

In Example 7, the medical system according to any of Examples 1-6, wherein exterior surfaces of the microelectrodes conform to an exterior surface of the metallic electrode to form an electrode assembly with a substantially continuous exterior surface.

In Example 8, the medical system according to any of Examples 1-7, wherein the metallic electrode has a cylindrical wall, a bore surrounded by the cylindrical wall, and a plurality of holes extending through the cylindrical wall in communication with the bore, and wherein the microelectrodes are respectively disposed within the holes.

In Example 9, the medical system according to any of Examples 1-8, further comprising a plurality of electrically insulative bands respectively disposed within the holes, wherein the microelectrodes are respectively disposed within the electrically insulative bands.

In Example 10, a medical system includes a medical probe including an elongated member having a distal end, a metallic electrode mounted to the distal end of the elongated member, a plurality of microelectrodes, and one or more ring electrodes proximal to the plurality of mapping microelectrode. The metallic electrode is configured to deliver RF ablation energy to tissue. A filter circuit is electrically connected to the microelectrodes such that the filter circuit receives electrical signals from the microelectrodes and mapping ring electrodes. The filter circuit is configured to filter components induced by the RF ablation energy from the electrical signals. A mapping processor electrically coupled to the filter circuit and configured to receive and process the filtered electrical signals.

In Example 11, the medical system according to Example 10, wherein the filter circuit comprises a plurality of filters each associated with one of the plurality of microelectrodes.

In Example 12, the medical system according to either Example 10 or Example 11, wherein the filter circuit is configured to filter frequency components from the electrical signals in the range of about 30-600 kHz.

In Example 13, the medical system according to any of Examples 10-12, wherein the filter circuit comprises one or more passive filters.

In Example 14, the medical system according to any of Examples 10-13, wherein the filter circuit comprises one or more digital filters.

In Example 15, the medical system according to any of Examples 10-14, wherein the plurality of microelectrodes are embedded within, and electrically insulated from, the metallic electrode.

In Example 16, a medical system is for use with a medical probe having an ablation electrode configured to deliver RF ablation energy and a plurality of microelectrodes embedded within, and electrically insulated from, the ablation electrode. The medical system includes a filter circuit configured to electrically connect to the microelectrodes such that the filter circuit receives electrical signals from the microelectrodes. The filter circuit is further configured to filter components induced by the RF ablation energy delivered by the metallic electrode from the electrical signals. A mapping processor is electrically coupled to the filter circuit and configured to receive the filtered electrical signals and output electrocardiograms based on the filtered electrical signals.

In Example 17, the medical system according to Example 16, wherein the filter circuit comprises a plurality of filters each associated with one of the plurality of microelectrodes.

In Example 18, the medical system according to either Example 16 or Example 17, wherein the filter circuit is configured to filter frequency components from the electrical signals in the range of about 30-600 kHz.

In Example 19, the medical system according to any of Examples 16-18, wherein the filter circuit comprises one or more passive filters.

In Example 20, the medical system according to any of Examples 16-19 wherein the filter circuit comprises one or more digital filters.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a plan view of one embodiment of an electrophysiology system constructed in accordance with the present disclosure.

FIG. 2 is a partially cutaway plan view of an electrophysiology catheter used in the system of FIG. 1, particularly showing a first arrangement of microelectrodes.

FIG. 3 is a cross-sectional view of the electrophysiology catheter of FIG. 2, taken along the line 3-3.

FIG. 4 is a cross-sectional view of one microelectrode incorporated into the electrophysiology catheter of FIG. 2.

FIG. 5 is a partially cutaway plan view of the electrophysiology catheter of FIG. 2, particularly showing a second arrangement of microelectrodes.

FIG. 6 is a partially cutaway plan view of the electrophysiology catheter of FIG. 2, particularly showing a third arrangement of microelectrodes.

FIG. 7 is a partially cutaway plan view of the electrophysiology catheter of FIG. 2, particularly showing a fourth arrangement of microelectrodes.

FIG. 8 is a partially cutaway plan view of the electrophysiology catheter of FIG. 2, particularly showing a fifth arrangement of microelectrodes.

FIG. 9 is a distal view of the electrophysiology catheter of FIG. 2.

FIG. 10 is a cross-sectional view of another microelectrode incorporated into the electrophysiology catheters of FIGS. 4 and 5.

FIGS. 11A-11C are plan views of a method of using the electrophysiology system of FIG. 1 to map and create lesions within the left atrium of a heart.

### DETAILED DESCRIPTION

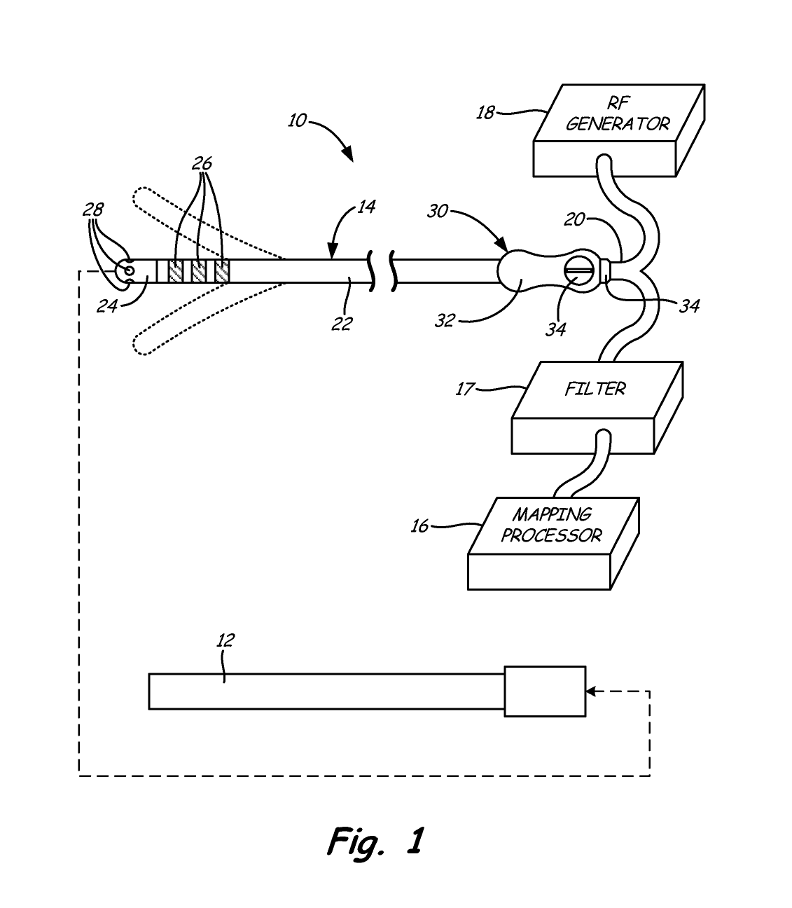

FIG. 1 illustrates an exemplary electrophysiology system 10 constructed in accordance with the present inventions is shown. The system 10 may be used within body lumens, chambers or cavities of a patient for therapeutic and diagnostic purposes in those instances where access to interior bodily regions is obtained through, for example, the vascular system or alimentary canal and without complex invasive surgical procedures. For example, the system 10 has application in the diagnosis and treatment of arrhythmia conditions within the heart. The system 10 also has application in the treatment of ailments of the gastrointestinal tract, prostrate, brain, gall bladder, uterus, and other regions of the body. As an example, the system 10 will be described hereinafter for use in the heart for mapping and ablating arrhythmia substrates.

The system 10 generally comprises a conventional guide sheath 12, and an electrophysiology catheter 14 that can be guided through a lumen (not shown) in the guide sheath 12. As will be described in further detail below, the electrophysiology catheter 14 is configured to be introduced through the vasculature of the patient, and into one of the chambers of the heart, where it can be used to map and ablate myocardial tissue. The system 10 also comprises a mapping processor 16, a mapping signal filter 17, and a source of ablation energy, and in particular, a radio frequency (RF) generator 18, coupled to the electrophysiology catheter 14 via a cable assembly 20. Although the mapping processor 16, mapping signal filter 17, and RF generator 18 are shown as discrete components, they can alternatively be combined in one or more integrated devices.

The mapping processor 16 is configured to detect, process, and record electrical signals within the heart via the electrophysiology catheter 14. Based on these electrical signals, a physician can identify the specific target tissue sites within the heart, and ensure that the arrhythmia causing substrates have been electrically isolated by the ablative treatment. Based on the detected electrical signals, the mapping processor 16 outputs electrocardiograms (EGMs) to a display (not shown), which can be analyzed by the user to determine the existence and/or location of arrhythmia substrates within the heart and/or determine the location of the electrophysiology catheter 14 within the heart. In an optional embodiment, the mapping processor 16 can generate and output an isochronal map of the detected electrical activity to the display for analysis by the user. Such mapping techniques are well known in the art, and thus for purposes of brevity, will not be described in further detail.

The mapping signal filter 17 is connected between the electrophysiology catheter 14 and mapping processor 16. The mapping signal filter 17 is configured to receive electrical signals from the electrophysiology catheter 14 and filter electrical signals to eliminate or reduce effects on the electrical signals caused by fields generated at the distal end of the electrophysiology catheter 14 by energy delivered from RF generator 18 during an ablation procedure. In some embodiments, the mapping signal filter 17 includes a printed circuit board or other assembly with filter circuits associated with each signal channel from the electrophysiology catheter 14. For example, the filters associated with each circuit may include RLC type filters or digital filters.

The RF generator 18 is configured to deliver ablation energy to the electrophysiology catheter 14 in a controlled manner in order to ablate the target tissue sites identified by the mapping processor 16. Ablation of tissue within the heart is well known in the art, and thus for purposes of brevity, the RF generator 18 will not be described in further detail. Further details regarding RF generators are provided in U.S. Pat. No. 5,383,874, which is expressly incorporated herein by reference.

The electrophysiology catheter 14 may be advanced though the guide sheath 12 to the target location. The sheath 12, which should be lubricious to reduce friction during movement of the electrophysiology catheter 14, may be advanced over a guidewire in conventional fashion. Alternatively, a steerable sheath may be provided. With respect to materials, the proximal portion of the sheath 12 may be a Pebax® material and stainless steel braid composite, and the distal portion is a more flexible material, such as unbraided Pebax®, for steering purposes. The sheath 12 should also be stiffer than the electrophysiology catheter 14. A sheath introducer (not shown), such as those used in combination with basket catheters, may be used when introducing the electrophysiology catheter 14 into the sheath 12. The guide sheath 12 may include a radio-opaque compound, such as barium, so that the guide sheath 12 can be observed using fluoroscopic or ultrasound imaging, or the like. Alternatively, a radio-opaque marker (not shown) can be placed at the distal end of the guide sheath 12.

The electrophysiology catheter 14 comprises an integrated flexible catheter body 22, a plurality of distally mounted electrodes, and in particular, a tissue ablation electrode 24, a plurality of mapping ring electrodes 26, a plurality of mapping microelectrodes 28, and a proximally mounted handle assembly 30. In alternative embodiments, the flexible catheter 14 may be replaced with a rigid surgical probe if percutaneous introduction or introduction through a surgical opening within a patient is desired.

The handle assembly 30 comprises a handle 32 composed of a durable and rigid material, such as medical grade plastic, and ergonomically molded to allow a physician to more easily manipulate the electrophysiology catheter 14. The handle assembly 30 comprises an external connector 34, such as an external multiple pin connector, received in a port on the handle assembly 30 with which the cable assembly 20 mates, so that the mapping processor 16 and RF generator 18 can be functionally coupled to the electrophysiology catheter 14. The handle assembly 30 may also include a printed circuit (PC) board (not shown) coupled to the external connector 34 and contained within the handle 32. The handle assembly 30 further including a steering mechanism 34, which can be manipulated to bidirectionally deflect the distal end of the electrophysiology catheter 14 (shown in phantom) via steering wires (not shown). Further details regarding the use of steering mechanisms are described in U.S. Pat. Nos. 5,254,088 and 6,579,278, which are expressly incorporated herein by reference in their entireties for all purposes.

The catheter body 22 may be about 5 French to 9 French in diameter, and between 80 cm to 150 cm in length. The catheter body 22 may have a cross-sectional geometry that is circular. However, other cross-sectional shapes, such as elliptical, rectangular, triangular, and various customized shapes, may be used as well. The catheter body 22 may be preformed of an inert, resilient plastic material that retains its shape and does not soften significantly at body temperature; for example, Pebax®, polyethylene, or Hytrel®) (polyester). Alternatively, the catheter body 22 may be made of a variety of materials, including, but not limited to, metals and polymers. The catheter body may be flexible so that it is capable of winding through a tortuous path that leads to a target site, i.e., an area within the heart. Alternatively, the catheter body 22 may be semi-rigid, i.e., by being made of a stiff material, or by being reinforced with a coating or coil, to limit the amount of flexing.

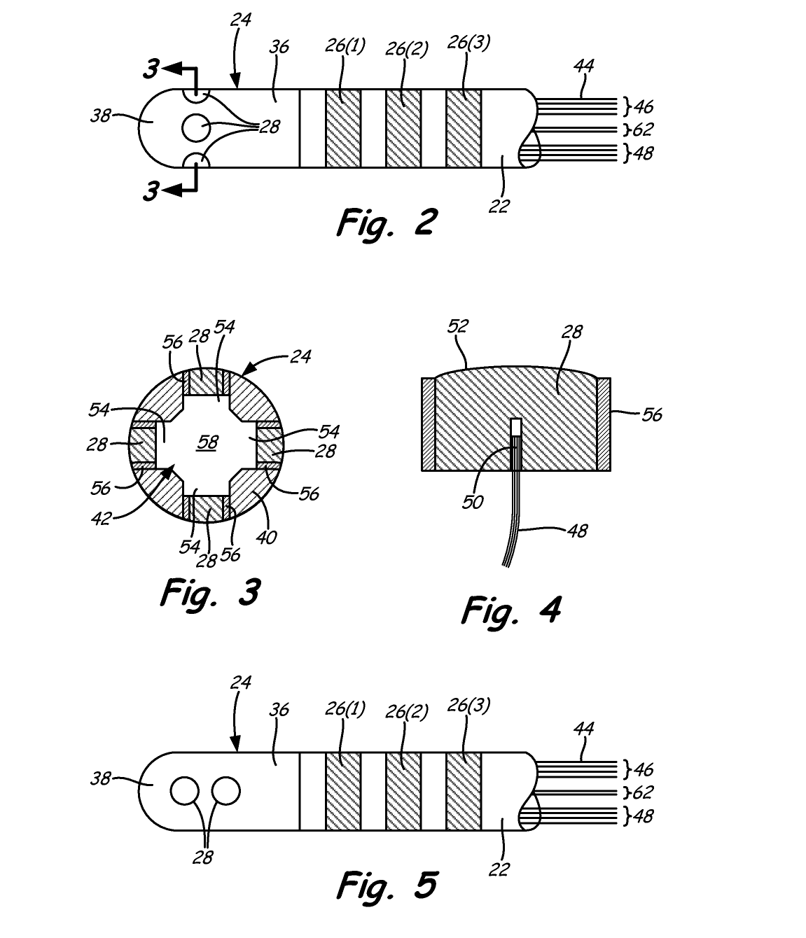

In the illustrated embodiment, the tissue ablation electrode 24 takes the form of a cap electrode mounted to the distal tip of the catheter body 22. In particular, and with further reference to FIG. 2, the ablation electrode 24 has a cylindrically-shaped proximal region 36 and a hemispherical distal region 38. As shown further in FIG. 3, the proximal region 36 of the ablation electrode 24 has a wall 40 and a bore 42 surrounded by the wall 40. The ablation electrode 24 may have any suitable length; for example, in the range between 4 mm and 10 mm. In the illustrated embodiment, the length of the ablation electrode 24 is 8 mm. The ablation electrode 24 may be composed of a solid, electrically conductive material, such as platinum, gold, or stainless steel. The wall 40 of the ablation electrode 24 has a suitable thickness, such that the ablation electrode 24 forms a rigid body. For the purposes of this specification, an electrode is rigid if it does not deform when pressed into firm contact with solid tissue (e.g., cardiac tissue). The ablation electrode 24 is electrically coupled to the RF generator 18 (shown in FIG. 1), so that ablation energy can be conveyed from the RF generator 18 to the ablation electrode 24 to form lesions in myocardial tissue. To this end, an RF wire 44 (shown in FIG. 2) is electrically connected to the ablation electrode 24 using suitable means, such as soldering or welding. The wire 44 is passed in a conventional fashion through a lumen (not shown) extending through the associated catheter body 22, where it is electrically coupled either directly to the external connector 34 or indirectly to the external connector 34 via the PC board located in the handle assembly 30, which, in turn, is electrically coupled to the RF generator 18 via the cable assembly 20.

The mapping ring electrodes 26 include a distal mapping ring electrode 26(1), a medial mapping ring electrode 26(2), and a proximal mapping ring electrode 26(3). The mapping ring electrodes 26, as well as the tissue ablation electrode 24, are capable of being configured as bipolar mapping electrodes. In particular, the ablation electrode 24 and distal mapping ring electrode 26(1) can be combined as a first bipolar mapping electrode pair, the distal mapping ring electrode 26(1) and the medial mapping ring electrode 26(2) may be combined as a second bipolar mapping electrode pair, and the medial mapping ring electrode 26(2) and the proximal mapping ring electrode 26(3) may be combined as a third bipolar mapping electrode pair.

In the illustrated embodiment, the mapping ring electrodes 26 are composed of a solid, electrically conducting material, like platinum, gold, or stainless steel, attached about the catheter body 22. Alternatively, the mapping ring electrodes 26 can be formed by coating the exterior surface of the catheter body 22 with an electrically conducting material, like platinum or gold. The coating can be applied using sputtering, ion beam deposition, or equivalent techniques. The mapping ring electrodes 26 can have suitable lengths, such as between 0.5 mm and 5 mm. The mapping ring electrodes 26 are electrically coupled to the mapping processor 16 (shown in FIG. 1), so that electrical events in myocardial tissue can be sensed for the creation of electrograms or monophasic action potentials (MAPs), or alternatively, isochronal electrical activity maps. To this end, signal wires 46 (shown in FIG. 2) are respectively connected to the mapping ring electrodes 26 using suitable means, such as soldering or welding. The signal wires 46 are passed in a conventional fashion through a lumen (not shown) extending through the associated catheter body 22, where they are electrically coupled either directly to the external connector 34 or indirectly to the external connector 34 via the PC board located in the handle assembly 30, which, in turn, is electrically coupled to the mapping electrode filter 17 via the cable assembly 20. Thus, each mapping ring electrode 26 provides signals to the mapping signal filter 17.

Like the mapping ring electrodes 26, the mapping microelectrodes 28 are electrically coupled to the mapping processor 16 (shown in FIG. 1), so that electrical events in myocardial tissue can be sensed for the creation of electrograms or MAPs, or alternatively, isochronal electrical activity maps. To this end, signal wires 48 (shown in FIG. 2) are respectively connected to the mapping microelectrodes 28 using suitable means, such as soldering or welding. The signal wires 48 are passed in a conventional fashion through a lumen (not shown) extending through the associated catheter body 22, where they are electrically coupled either directly to the external connector 34 or indirectly to the external connector 34 via the PC board located in the handle assembly 30, which, in turn, is electrically coupled to the mapping electrode filter 17 via the cable assembly 20. Thus, each mapping microelectrode 28 provides signals to the mapping signal filter 17.

Significantly, the microelectrodes 28 are disposed on the tissue ablation electrode 24, and in particular, are embedded within the wall 40 of the tissue ablation electrode 24. This allows the localized intracardial electrical activity to be measured in real time at the point of energy delivery from the ablation electrode 24. In addition, due to their relatively small size and spacing, the microelectrodes 28 do not sense far field electrical potentials that would normally be associated with bipolar measurements taken between the tissue ablation electrode 24 and the mapping ring electrodes 26.

Instead, the microelectrodes 28 measure the highly localized electrical activity at the point of contact between the ablation electrode 24 and the endocardial tissue. Thus, the arrangement of the microelectrodes 28 substantially enhances the mapping resolution of the electrophysiology catheter 14. The high resolution inherent in the microelectrode arrangement will allow a user to more precisely measure complex localized electrical activity, resulting in a powerful tool for diagnosing EGM activity; for example, the high frequency potentials that are encountered around pulmonary veins or the fractioned EGMs associated with atrial fibrillation triggers.

As discussed above, electric fields generated by the ablation electrode 24 during an ablation procedure may affect the signals sensed by the mapping ring electrodes 26 and mapping microelectrodes 28. In order to reduce or eliminate effects of these fields on the mapping electrode signals, the mapping signal filter 17 is connected between the electrophysiology catheter 14 and mapping signal processor 16. The mapping signal filter 17 is configured to filter signal components caused by the RF energy provided to the ablation electrode 24. For example, RF generators, such as RF generator 18, may operate at frequencies around 500 kHz. In order to reduce or eliminate the electrical noise picked up by the mapping ring electrodes 26 and mapping electrodes 28 from the ablation electrode 24, the mapping signal filter 17 is configured to filter signal components around the operating frequency (e.g., 500 kHz) of the RF generator 18. In some embodiments, because the mapping electrodes 26, 28 record signals at frequencies well outside the typical operating frequencies of RF generators (e.g., 40-100 Hz), the mapping signal filter 17 is configured to filter a large range of frequencies around the operating frequency of the RF generator 18. For example, in some embodiments, the mapping signal filter 17 is configured to filter signals in the range of about 30-600 kHz. As noted above, each mapping electrode 26, 28 may be connected to a filter that filters a range of frequencies around the operating frequency of the RF generator 18. In some embodiments, the filters in the mapping signal filter 17 are passive circuits (e.g., RLC circuits) that are configured as bandstop or low-pass filters. In other embodiments, the filters in the mapping signal filter 17 are digital filters.

In some embodiments, the filtered components of the mapping electrode signals are provided to a reference ground loop to which the mapping signal filter 17 is attached.

The microelectrode arrangement lends itself well to creating MAPs, which may play an important role in diagnosing AFIB triggers. In particular, a focal substrate may be mapped by the microelectrodes 28, and without moving the ablation electrode 24, the mapped focal substrate may be ablated. The microelectrode arrangement also allows for the generation of high density electrical activity maps, such as electrical activity isochronal maps, which may be combined with anatomical maps, to create electro-anatomical maps. In addition, due to the elimination or minimization of the detected far field electrical activity, detection of tissue contact and tissue characterization, including lesion formation assessment, is made more accurate.

The microelectrodes 28 may be disposed on the ablation electrode 24 in any one of a variety of different patterns. In the embodiment illustrated in FIG. 2, four microelectrodes 28 (only three shown) are circumferentially disposed about the cylindrical-shaped region 36 of the ablation electrode 24 at ninety degree intervals, so that they face radially outward in four different directions. In another embodiment illustrated in FIG. 5, four microelectrodes 28 are arranged into two longitudinally disposed pairs (only pair shown) circumferentially disposed about the cylindrical-shaped proximal region 36 of the ablation electrode 24 at a one hundred degree interval, so that the electrode pairs face radially outward in two opposite directions.

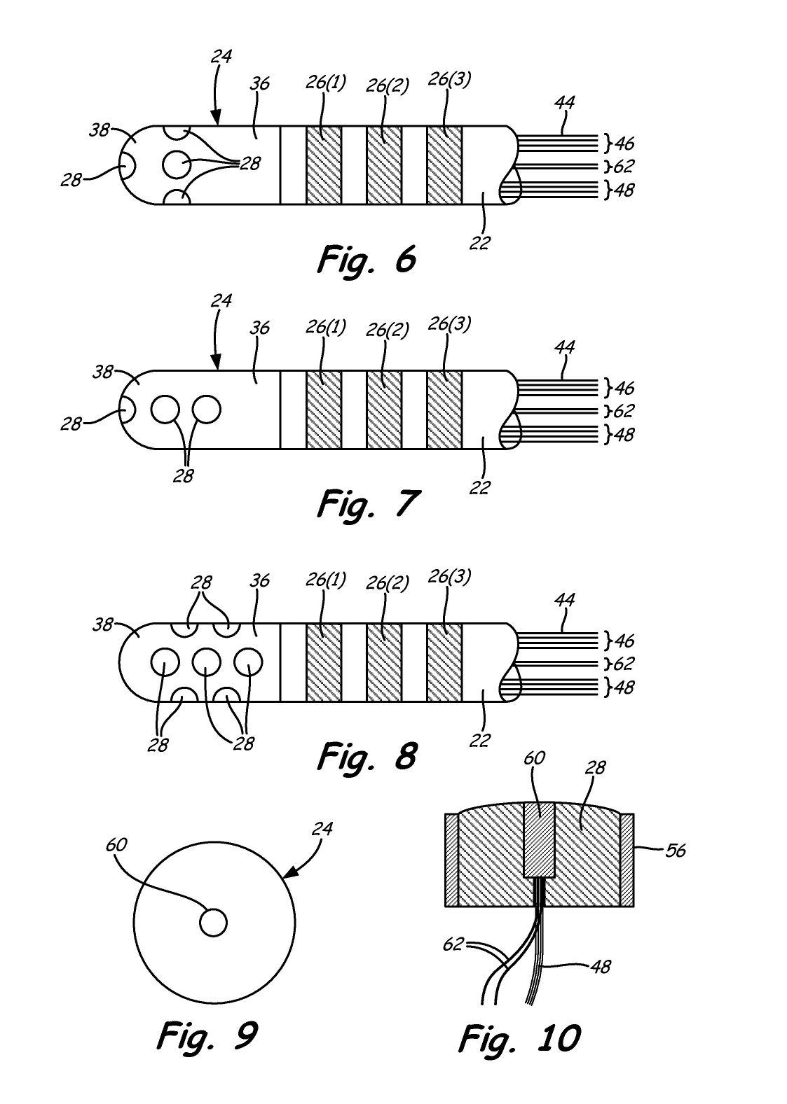

Other embodiments illustrated in FIGS. 6 and 7, are respectively similar to the embodiments illustrated in FIGS. 4 and 5, with the exception that a fifth microelectrode 28 is disposed on the hemispherical distal region 38 of the ablation electrode 24, so that it faces distally outward. In yet another embodiment, as shown in FIG. 8, ten microelectrodes 28 are arranged into two longitudinally disposed trios (only one shown) and two longitudinally disposed pairs circumferentially disposed about the cylindrical-shaped proximal region 36 of the ablation electrode 24 at ninety degree intervals, so that the electrode trios and pairs face radially outward in four different directions. Notwithstanding the different microelectrode patterns, in some embodiments, the microelectrodes 28 are located as distal on the ablation electrode 24 as possible. In this manner, the microelectrodes 28 will be placed into contact with tissue when the distal end of the electrophysiology catheter 14 is oriented perpendicularly to the tissue.

In the illustrated embodiments, each of the microelectrodes 28 has a circular profile for ease of manufacture, although in alternative embodiments, the microelectrodes 28 may have other profiles, such as elliptical, oval, or rectangular. The microelectrodes 28 have relatively small diameters and are spaced a relatively small distance from each other in order to maximize the mapping resolution of the microelectrodes 28, as will be described in further detail below. Ultimately, the size and spacing of the microelectrodes 28 will depend upon the size of the ablation electrode 24, as well as the number and particular pattern of the microelectrodes 28. In some embodiments, the diameter of each microelectrode 28 is equal to or less than half the length of the ablation electrode 24. For example, in some embodiments, the diameter of each microelectrode 28 is equal to or less than one-quarter the length of the ablation electrode 24. For example, if the length of the ablation electrode 24 is 8 mm, the diameter of each microelectrode 28 may be equal to or less than 4 mm, for example equal to or less than 2 mm. The spacing of the microelectrodes 28 (as measured from center to center) may be equal to or less than twice the diameter, for example equal to or less than one and half times the diameter of each microelectrode 28.

Each microelectrode 28 is composed of an electrically conductive material, such as platinum, gold, or stainless steel. In some embodiments, each microelectrode 28 is composed of a silver/silver chloride to maximize the coupling between the microelectrode 28 and blood, thereby optimizing signal fidelity. As shown in FIG. 4, each microelectrode 28 is substantially solid, having a small bore 50 formed in one end of the microelectrode 28 along its axis, thereby providing a convenient means for connecting a signal wire 48 to the microelectrode 28 via suitable means, such as soldering or welding.

Each microelectrode 28 also has a tissue-contacting surface 52 opposite the bore 42 that may conform with the tissue-contacting surface of the ablation electrode 24. Thus, because the tissue-contacting surface of the ablation electrode 24 is curved, the tissue-contacting surface 52 of each microelectrode 28 is likewise curved, with the radii of curvature for the respective surface being the same, thereby forming an electrode assembly with a substantially continuous surface (i.e., a surface with very little discontinuities or sharp edges). In this manner, RF energy will not be concentrated within localized regions of the ablation electrode 24 to create “hot spots” that would undesirably char tissue, which may otherwise occur at discontinuities. Alternatively, the tissue-contacting surface 52 of each microelectrode 28 may have a flat surface tangent to the curvature of the ablation tip. To ensure that the electrode assembly has a continuous external surface, the exterior surfaces of the ablation electrode 24 and microelectrodes 28 can be ground to a fine finish (e.g., #16 rms). The fine finish also contributes to signal fidelity and acts as a thrombus inhibitor.

Referring to FIG. 3, the ablation electrode 24 comprises a plurality of holes 54 laterally extending through the wall 40 in communication with the bore 42, and the microelectrodes 28 are respectively disposed in the holes 54. The holes 54 may be formed by drilling through the wall 40 of the ablation electrode 24. Significantly, the microelectrodes 28 are electrically insulated from the ablation electrode 24, and thus, from each other, so that they can provide independent mapping channels. The microelectrodes 28 are also thermally insulated from the ablation electrode 24 to prevent saturation of the mapping channels that would otherwise cause interference from the heat generated during a radio frequency (RF) ablation procedure.

To this end, the ablation electrode 24 comprises a plurality of insulative bands 56 (best shown in FIG. 4) composed of the suitable electrically and thermally insulative material, such as a high temperature thermoset plastic with high dielectric properties, e.g., polyimide or plastics from the phenolic group, such as Bakelite® or Ultem® plastics. The insulative bands 56 are respectively mounted within the holes 54, and the microelectrodes 28 are mounted in the insulative bands 56. In this manner, the insulative bands 56 are interposed between the wall 40 of the ablation electrode 24 and the microelectrodes 28 to provide the desirable electrical and thermal insulation. The insulative bands 56 and microelectrodes 28 may be respectively mounted within the holes 54 using a suitable bonding material, such as, epoxy. An electrically and thermally insulative potting material 58 (such as a multicomponent (resin and hardener component) thermosetting or ultra-violet (UV)-curable resin, for example, silicone, urethane or epoxy) can also be introduced into the bore 42 of the ablation electrode 24 to ensure electrical insulation between the microelectrodes 28 and ablation electrode 24, to further secure the microelectrodes 28 to the ablation electrode 24, and to prevent cross-talk between the otherwise electrically insulated microelectrodes 28. In some embodiments, the radii of the insulative bands 56 are configured to blend into the ablation electrode 24 to reduce potential current concentrations. In alternative embodiments, the microelectrodes 28 are deposited or formed on an exterior surface of the ablation electrode 24.

The electrophysiology catheter 14 further comprises a temperature sensor 60, such as a thermocouple or thermistor, which may be located on, under, abutting the longitudinal end edges of, or in the ablation electrode 24. In the illustrated embodiment, the temperature sensor 60 is mounted within a bore 42 formed at the distal tip of, and along the longitudinal axis of, the ablation electrode 24, as illustrated in FIG. 9, or, if a microelectrode 28 is incorporated into the distal tip of the ablation electrode 24, as illustrated in FIGS. 6 and 7, within a bore 42 formed within, and along the longitudinal axis of, a microelectrode 28, as illustrated in FIG. 10. For temperature control purposes, signals from the temperature sensors are transmitted to the RF generator 18 via signal wires 62, so that RF energy to the ablation electrode 24 may be controlled based on sensed temperature. To this end, the signal wires 62 are passed in a conventional fashion through a lumen (not shown) extending through the associated catheter body 22, where they are electrically coupled either directly to the external connector 34 or indirectly to the external connector 34 via the PC board located in the handle assembly 30, which, in turn, is electrically coupled to the RF generator 18 via the cable assembly 20.

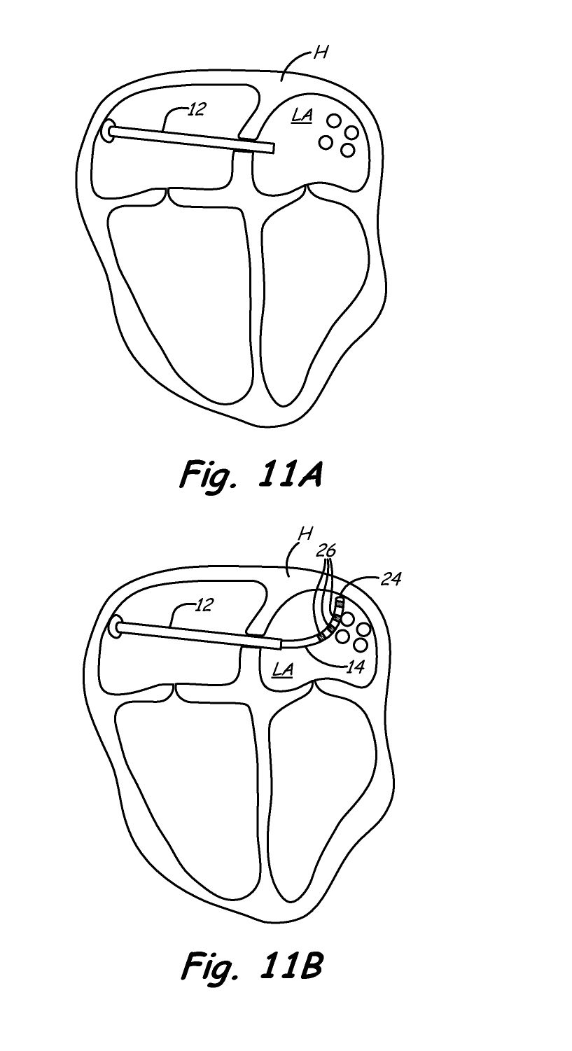

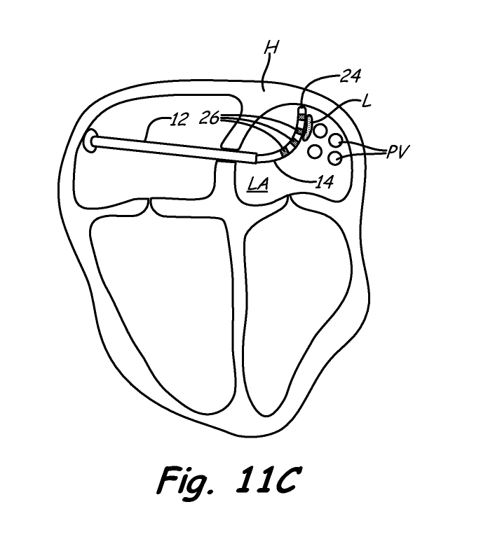

Having described the structure of the medical system 10, its operation in creating a lesion within the left atrium LA of the heart H to ablate or electrically isolate arrhythmia causing substrates will now be described with reference to FIGS. 11A-11C. It should be noted that other regions within the heart H can also be treated using the medical system 10. It should also be noted that the views of the heart H and other interior regions of the body described herein are not intended to be anatomically accurate in every detail. The figures show anatomic details in diagrammatic form as necessary to show the features of the embodiment described herein.

First, the guide sheath 12 is introduced into the left atrium LA of the heart H, so that the distal end of the sheath 12 is adjacent a selected target site (FIG. 11A). Introduction of the guide sheath 12 within the left atrium LA can be accomplished using a conventional vascular introducer retrograde through the aortic and mitral valves, or can use a transeptal approach from the right atrium, as illustrated in FIG. 11A. A guide catheter or guide wire (not shown) may be used in association with the guide sheath 12 to aid in directing the guide sheath 12 through the appropriate artery toward the heart H.

Once the distal end of the guide sheath 12 is properly placed, the electrophysiology catheter 14 is introduced through the guide sheath 12 until its distal end is deployed from the guide sheath 12 (FIG. 11B). The steering mechanism 34 located on the handle assembly 30 (shown in FIG. 1) may be manipulated to place the ablation electrode 24 into firm contact with the endocardial tissue at a perpendicular angle to the wall of the heart H.

Once the ablation electrode 24 is firmly and stably in contact with the endocardial tissue, the mapping processor 16 (shown in FIG. 1) is operated in order to obtain and record EGM or MAP signals from the myocardial tissue via bipolar pairs of the microelectrodes 28 (shown in FIG. 1). These EGM or MAP signal measurements can be repeated at different locations within the left atrium LA to ascertain one or more target sites to be ablated. The user can analyze the EGMs or MAPs in a standard manner, or if electrical activity isochronal maps (whether or not combined with anatomical maps), can analyze these, to ascertain these target sites. Significantly, the use of the microelectrodes 28 substantially increases the resolution and enhances the fidelity of the EGM or MAP measurements. Alternatively, the mapping processor 16 can be operated to obtain and record EGM or MAP signals from the myocardial tissue via bipolar pairs of the ablation electrode 24 and mapping ring electrodes 26 if far field electrical potentials are desired; that is generalized mapping, in addition to highly localized mapping is desired.

Once a target site has been identified via analysis of the EGM or MAP signals or isochronal electrical activity maps, the ablation electrode 24 is placed into firm contact with the target site, and the RF generator 18 (shown in FIG. 1) is then operated in order to convey RF energy to the ablation electrode 24 (either in the monopolar or bipolar mode), thereby creating a lesion L (FIG. 11C). Firm contact between the ablation electrode 24 and the endocardial tissue of the heart H can be confirmed by analyzing the EGM or MAP signals measured by the microelectrodes 28, with the amplitude of the EGM or MAP signals increasing as contact between the ablation electrode 24 and the endocardial tissue increases.

In the case where ablation is performed in or around the ostia PV of blood vessels, such as pulmonary veins or the superior vena cava, the contact with the endocardial tissue, as opposed to venous tissue, can be confirmed via analysis of the highly localized EGM or MAP signals measured by the microelectrodes 28. Ablation of the target site can be confirmed, again, by analyzing the highly localized EGM or MAP signals measured by the microelectrodes 28 during and after the ablation procedure, with the amplitude of the EGM or MAP signals gradually decreasing to zero as the tissue is successfully ablating. Significantly, since the microelectrodes 28 are incorporated into the ablation electrode 24, target site identification, electrode-tissue contact and characterization, tissue ablation, and lesion confirmation can all be performed without moving the ablation electrode 24.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A medical system, comprising:
a medical probe including an elongated member having a distal end, a metallic electrode mounted to the distal end of the elongated member, and a plurality of microelectrodes embedded within, and electrically insulated from, the metallic electrode;
a radio frequency (RF) ablation source configured to deliver RF ablation energy to the metallic electrode;
a filter circuit electrically connected to the microelectrodes such that the filter circuit receives electrical signals from the microelectrodes, wherein the filter circuit is configured to filter signal components induced by the RF ablation energy from the electrical signals; and
a mapping processor electrically coupled to the filter circuit and configured to receive and process the filtered electrical signals.

2. The medical system of claim 1, wherein the filter circuit comprises a plurality of filters each associated with one of the plurality of microelectrodes.

3. The medical system of claim 1, wherein the filter circuit is configured to filter frequency components from the electrical signals in the range of about 30-600 kHz.

4. The medical system of claim 1, wherein the filter circuit comprises one or more passive filters.

5. The medical system of claim 1, wherein the filter circuit comprises one or more digital filters.

6. The medical system of claim 1, wherein the medical probe further comprises one or more mapping ring electrodes, and wherein the one or more mapping ring electrodes are electrically connected to the filter circuit to filter RF ablation energy induced components from signals from the mapping ring electrodes.

7. The medical system of claim 1, wherein exterior surfaces of the microelectrodes conform to an exterior surface of the metallic electrode to form an electrode assembly with a substantially continuous exterior surface.

8. The medical system of claim 1, wherein the metallic electrode has a cylindrical wall, a bore surrounded by the cylindrical wall, and a plurality of holes extending through the cylindrical wall in communication with the bore, and wherein the microelectrodes are respectively disposed within the holes.

9. The medical system of claim 8, further comprising a plurality of electrically insulative bands respectively disposed within the holes, wherein the microelectrodes are respectively disposed within the electrically insulative bands.

10. A medical system, comprising:
a medical probe including an elongated member having a distal end, a metallic electrode mounted to the distal end of the elongated member, a plurality of microelectrodes, and one or more ring electrodes proximal to the plurality of mapping microelectrode, the metallic electrode configured to deliver RF ablation energy to tissue;
a filter circuit electrically connected to the microelectrodes such that the filter circuit receives electrical signals from the microelectrodes and mapping ring electrodes, wherein the filter circuit is configured to filter components induced by the RF ablation energy from the electrical signals; and
a mapping processor electrically coupled to the filter circuit and configured to receive and process the filtered electrical signals.

11. The medical system of claim 10, wherein the filter circuit comprises a plurality of filters each associated with one of the plurality of microelectrodes.

12. The medical system of claim 10, wherein the filter circuit is configured to filter frequency components from the electrical signals in the range of about 30-600 kHz.

13. The medical system of claim 10, wherein the filter circuit comprises one or more passive filters.

14. The medical system of claim 10, wherein the filter circuit comprises one or more digital filters.

15. The medical system of claim 10, wherein the plurality of microelectrodes are embedded within, and electrically insulated from, the metallic electrode.

16. A medical system for use with a medical probe having an ablation electrode configured to deliver RF ablation energy and a plurality of microelectrodes embedded within, and electrically insulated from, the ablation electrode, the medical system comprising:
a filter circuit configured to electrically connect to the microelectrodes such that the filter circuit receives electrical signals from the microelectrodes, wherein the filter circuit is further configured to filter components induced by the RF ablation energy delivered by the metallic electrode from the electrical signals; and
a mapping processor electrically coupled to the filter circuit and configured to receive the filtered electrical signals and output electrocardiograms based on the filtered electrical signals.

17. The medical system of claim 16, wherein the filter circuit comprises a plurality of filters each associated with one of the plurality of microelectrodes.

18. The medical system of claim 16, wherein the filter circuit is configured to filter frequency components from the electrical signals in the range of about 30-600 kHz.

19. The medical system of claim 16, wherein the filter circuit comprises one or more passive filters.

20. The medical system of claim 16, wherein the filter circuit comprises one or more digital filters.
